(19) United States
(12) Patent Application Publication
(10) Pub. No.: US 2019/0016646 A1
(43) Pub. Date: Jan. 17, 2019

(54) PEROVSKITE-CATALYZED HYDROGENOLYSIS OF HETEROATOM-CONTAINING COMPOUNDS

(71) Applicants: Massachusetts Institute of Technology, Cambridge, MA (US); BP America, Inc., Warrenville, IL (US)

(72) Inventors: Yuriy Román-Leshkov, Cambridge, MA (US); Manish Shetty, Cambridge, MA (US); William H. Green, Belmont, MA (US); Casey Hetrick, Plainfield, IL (US); John Shabaker, Naperville, IL (US)

(73) Assignees: Massachusetts Institute of Technology, Cambridge, MA (US); BP America, Inc., Warrenville, IL (US)

(21) Appl. No.: 15/648,407

(22) Filed: Jul. 12, 2017

(51) Int. Cl. C07C 1/20; C07C 1/32; B01J 23/00; B01J 23/83

(52) U.S. Cl. CPC C07C 1/20; C07C 1/323; B01J 2523/00; B01J 23/83; C07C 2523/83; B01J 23/002

(57) ABSTRACT

Perovskite compounds that catalyze hydrogenolysis (e.g., hydrodeoxygenation, hydrodenitrogenation, and/or hydrodesulfurization) of heteroatom-containing compounds, as well as associated systems and methods, are generally described. In some embodiments, methods are provided for contacting a perovskite compound with a heteroatom-containing compound (e.g., a compound comprising oxygen, nitrogen, and/or sulfur) in the presence of hydrogen gas (H₂) such that the perovskite compound catalyzes hydrogenolysis of the heteratom-containing compound to produce one or more hydrocarbon products (e.g., one or more aromatic hydrocarbons and/or aliphatic hydrocarbons). According to certain embodiments, the perovskite compound has the formula A₁₋ₓBₓDO₃, where A comprises a lanthanide, B comprises an alkaline earth metal, D comprises a transition metal, and x is greater than or equal to 0 and less than or equal to 1. Compounds, systems, and methods described herein may be useful for applications involving petroleum (e.g., crude oil) and/or biofuels.

### FIELD

The present invention generally relates to perovskite-catalyzed hydrogenolysis of heteroatom-containing compounds.

### BACKGROUND

As energy needs continue to increase around the world, there is heightened demand for both petroleum (e.g., crude oil) and biofuels. Given this heightened demand, there is a pressing need for methods of removing heteroatoms from carbonaceous materials such as crude oil and biomass. For example, a considerable amount of effort has been dedicated to the removal of heteroatom impurities from crude oil in order to minimize the negative impact of pollutants. As a result, hydrodenitrogenation (HDN) and hydrodesulfurization (HDS) reactions have been employed to help reduce toxic SO₂ and NOₓ emissions resulting from fuel combustion and to remove sulfur and nitrogen impurities that are catalyst poisons for other chemical transformations in petrochemical refineries. In addition, hydrodeoxygenation (HDO) reactions have been investigated to reduce the oxygen content of biomass and thereby facilitate the production of sustainable biofuels.

Current industrial catalysts of HDX reactions (X=O, N, and/or S) often include molybdenum or tungsten. These catalysts may be supported (e.g., on high-surface-area supports such as γ-Al₂O₃, acidic supports such as SiO₂—Al₂O₃, zeolites, and/or neutral supports such as carbon) or unsupported, and they may be promoted by one or more transition metals (e.g., Ni, Co). For example, one state-of-the-art catalyst is NEBULA® (New Bulk Activity), an unsupported base metal catalyst of NiMoW that was jointly developed by ExxonMobil and

Albemarle. In light of stricter emission regulations, an increased focus on renewable energy, and the properties of current catalysts, however, it is desirable to develop new families of catalysts with high catalytic activity towards HDX reactions. In particular, it is desirable to develop catalysts for HDX reactions that exhibit high catalytic activity at lower temperatures and pressures than the current industrial catalysts. Accordingly, improved HDX catalysts are needed.

### SUMMARY

The present invention generally relates to perovskite-catalyzed hydrogenolysis of heteroatom-containing compounds. The subject matter of the present invention involves, in some cases, interrelated products, alternative solutions to a particular problem, and/or a plurality of different uses of one or more systems and/or articles.

Some aspects relate to a method. In some embodiments, the method comprises contacting a perovskite compound with a heteroatom-containing compound in the presence of H₂. In certain embodiments, the perovskite compound catalyzes hydrogenolysis of the heteroatom-containing compound to produce one or more hydrocarbon products.

In some embodiments, the method comprises introducing a first feed stream comprising a heteroatom-containing compound into a reactor comprising a catalyst bed comprising a perovskite compound. In some embodiments, the method further comprises introducing a second feed stream comprising H₂ into the reactor. In some embodiments, the method further comprises flowing the first feed stream and the second feed stream, or a mixed feed stream comprising a mixture of the first feed stream and the second feed stream, through the reactor. In certain embodiments, the first feed stream, the second feed stream, and/or the mixed feed stream directly contact the catalyst bed. In certain embodiments, the perovskite compound catalyzes a hydrogenolysis reaction to produce one or more product streams comprising one or more hydrocarbon compounds.

Some aspects relate to a system. In some embodiments, the system comprises a first feed stream comprising a heteroatom-containing compound. In some embodiments, the system comprises a second feed stream comprising H₂. In some embodiments, the system comprises a perovskite compound. In some embodiments, the system comprises one or more product streams comprising one or more hydrocarbon compounds.

Some aspects relate to a perovskite compound having the formula A₁₋ₓ,BₓDO₃. In some embodiments, A comprises a lanthanide. In some embodiments, B comprises an alkaline earth metal. In some embodiments, D comprises a transition metal. In some embodiments, x is greater than or equal to 0 and less than or equal to 1. In certain embodiments, the perovskite compound catalyzes hydrodenitrogenation, hydrodeoxygenation, and/or hydrodesulfurization reactions.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic diagram of an exemplary system comprising a reactor comprising a catalyst bed comprising pellets of a perovskite compound, a first feed stream comprising a heteroatom-containing compound, a second feed stream comprising hydrogen gas, and a product stream comprising one or more hydrocarbon compounds, according to some embodiments;

FIG. 2 shows, according to some embodiments, an exemplary representation of the hydrodeoxygenation of anisole catalyzed by La_{0.8}Sr_{0.2}CoO₃;

FIG. 3A shows a plot of the conversion (carbon-mol %) of anisole catalyzed by La_{0.8}Sr_{0.2}CoO₃as a function of time-of-stream (hours), according to some embodiments;

FIG. 3B shows plots of selectivity (carbon-mol%) for benzene (top), alkanes and cycloalkanes (middle), and substituted aromatics and substituted cycloalkanes (bottom) as a function of time-of-stream (hours) during the hydrodeoxygenation of anisole catalyzed by La_{0.8}Sr_{0.2}CoO₃, according to some embodiments;

FIG. 4 shows, according to some embodiments, an exemplary representation of the hydrodenitrogenation of pyridine catalyzed by La_{0.8}Sr_{0.2}CoO₃;

FIG. 5A shows a plot of the rate of hydrodenitrogenation (HDN) products as a function of time-of-stream (hours) during the hydrodenitrogenation of pyridine catalyzed by La_{0.8}Sr_{0.2}CoO₃, according to some embodiments;

FIG. 5B shows a chart of the selectivity (carbon-mol %) for C₁-C₃ alkanes, butane, pentane, and hexane during the hydrodenitrogenation of pyridine catalyzed by La_{0.8}Sr_{0.2}CoO₃, according to some embodiments;

FIG. 6 shows, according to some embodiments, a plot of selectivity/conversion (carbon-mol %) for benzene (top) and C₁-C₅ alkanes and cycloalkanes (bottom) as a function of time-of-stream (hours) during the hydrodeoxygenation of anisole catalyzed by LaNiO₃; and

FIG. 7 shows, according to some embodiments, a plot of selectivity/conversion (carbon-mol %) for benzene (top) and C₁-C₅ alkanes and cycloalkanes (bottom) as a function of time-of-stream (hours) during the hydrodeoxygenation of anisole catalyzed by La_{0.75}Sr_{0.25}CoO₃.

### DETAILED DESCRIPTION

Perovskite compounds that catalyze hydrogenolysis (e.g., hydrodeoxygenation, hydrodenitrogenation, and/or hydrodesulfurization) of heteroatom-containing compounds, as well as associated systems and methods, are generally described. In some embodiments, methods are provided for contacting a perovskite compound with a heteroatom-containing compound (e.g., a compound comprising oxygen, nitrogen, and/or sulfur) in the presence of hydrogen gas (H₂) such that the perovskite compound catalyzes hydrogenolysis of the heteratom-containing compound to produce one or more hydrocarbon products (e.g., one or more aromatic hydrocarbons and/or aliphatic hydrocarbons). According to certain embodiments, the perovskite compound has the formula A₁₋ₓ,BₓDO₃, where A comprises a lanthanide, B comprises an alkaline earth metal, D comprises a transition metal, and x is greater than or equal to 0 and less than or equal to 1. Compounds, systems, and methods described herein may be useful for applications involving petroleum (e.g., crude oil) and/or biofuels.

In some cases, it may be desirable to remove heteroatoms from certain carbonaceous materials, such as crude oil and biomass-derived products (e.g., bio-oil). For example, removal of sulfur from crude oil through a hydrodesulfurization reaction may advantageously reduce sulfur dioxide (SO₂) emissions from fuel combustion, and removal of nitrogen from crude oil through a hydrodenitrogenation reaction may advantageously reduce emission of nitrogen oxides (NOₓ) from fuel combustion. Removal of oxygen from biomass-derived products (e.g., bio-oil obtained from pyrolysis of biomass) may also be desirable, as biomass and biomass-derived products typically have high levels of oxygen that are often associated with various disadvantages (e.g., non-volatility, corrosiveness, thermal instability). In some cases, removal of oxygen from biomass (i.e., material comprising or derived from organic matter, such as plant-based materials) or biomass-derived products, such as bio-oil, through a hydrodeoxygenation reaction may advantageously convert the biomass and/or biomass-derived products into high-quality hydrocarbon fuel.

The inventors have unexpectedly found that perovskite compounds can catalyze hydrogenolysis (e.g., hydrodeoxygenation, hydrodenitrogenation, and/or hydrodesulfurization) of heteroatom-containing compounds. These perovskite compounds may be associated with certain advantages over existing hydrogenolysis catalysts. In some cases, for example, the perovskite compounds may exhibit high catalytic activity at relatively low temperatures and/or pressures. The ability to conduct hydrogenolysis reactions at relatively low temperatures and/or pressures may advantageously reduce costs. For example, in an oil refinery, high pressures may be associated with high capital costs (e.g., equipment that can withstand high pressures may be expensive), and the use of perovskite compounds as hydrogenolysis catalysts may result in substantial cost savings compared to existing hydrogenolysis catalysts.

In some cases, perovskite compounds exhibit high sulfur tolerance. This ability to maintain high catalytic activity in the presence of high sulfur concentrations may advantageously allow perovskite compounds to catalyze hydrogenolysis (e.g., hydrodeoxygenation, hydrodenitrogenation, and/or hydrodesulfurization) of heteroatom-containing compounds in feed streams with high sulfur content, such as certain crude oil feed streams. In addition, in some cases, the high sulfur tolerance of perovskite compounds may advantageously avoid the need for a sulfidation unit for catalyst preparation and regeneration.

In certain cases, perovskite compounds exhibit high selectivity for certain hydrocarbon products. For example, certain perovskite compounds may exhibit high selectivity for certain aromatic hydrocarbons. This high selectivity for certain aromatic hydrocarbons may, in some cases, advantageously preserve the aromaticity of certain feed streams (e.g., feed streams comprising nitrogen-containing aromatics, such as crude oil feed streams). In certain cases, perovskite compounds may prevent hydrogenation of aromatic hydrocarbons to aliphatic or cycloaliphatic hydrocarbons. In contrast, conventional hydrogenolysis catalysts (e.g., Ni—or Co—promoted, Mo—or W—supported sulfides) are often active for hydrogenation, resulting in H₂ consumption and formation of aliphatic and/or cycloaliphatic hydrocarbons.

Perovskite compounds may also be associated with certain other advantages, including, but not limited to, high thermal stability and ability to catalyze hydrogenolysis of a broad range of heteroatom-containing compounds.

Certain embodiments described herein are related to a perovskite compound. In some embodiments, the perovskite compound has the formula A₁₋ₓBₓDO₃, where “A,” “B,” and “D” each represent one or more elements. In certain embodiments, the “A,” “B,” and/or “D” sites are cationic.

In some embodiments, “A” comprises a lanthanide. As used herein, the term “lanthanide” refers to an element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). In certain embodiments, the lanthanide is La. In some embodiments, “A” comprises two or more lanthanides.

In some embodiments, “B” comprises an alkaline earth metal. As used herein, the term “alkaline earth metal” refers to an element selected from the group consisting of beryllium (Be), magnesium (Mg), calcium (Ca), strontium (Sr), barium (Ba), and radium (Ra). In certain embodiments, the alkaline earth metal is selected from the group consisting of Mg, Ca, Sr, and Ba. In certain embodiments, the alkaline earth metal is Sr. In some embodiments, “B” comprises two or more alkaline earth metals. As an illustrative, non-limiting example, “B” may comprise Ba and Sr.

In some embodiments, “D” comprises a transition metal. As used herein, the term “transition metal” refers to an element in the d-block of the periodic table, including elements from column 3 through column 12. Non-limiting examples of suitable transition metals include chromium (Cr), manganese (Mn), iron (Fe), cobalt (Co), and nickel (Ni). In certain embodiments, the transition metal is Co or Ni. In some embodiments, “D” comprises two or more transition metals. As illustrative, non-limiting examples, “D” may comprise Co and Fe or Co and Ni.

In some embodiments, the value of x in the formula A₁₋ₓ,BₓDO₃ is greater than or equal to about 0 and less than or equal to about 1.0. In some embodiments, the value of x is at least about 0, at least about 0.10, at least about 0.20, at least about 0.25, at least about 0.30, at least about 0.40, at least about 0.50, at least about 0.60, at least about 0.70, at least about 0.75, at least about 0.80, at least about 0.90, at least about 0.95, at least about 0.99, or about 1.0. In some embodiments, the value of x is less than or equal to about 1.0, less than or equal to about 0.99, less than or equal to about 0.95, less than or equal to about 0.90, less than or equal to about 0.80, less than or equal to about 0.75, less than or equal to about 0.70, less than or equal to about 0.60, less than or equal to about 0.50, less than or equal to about 0.40, less than or equal to about 0.30, less than or equal to about 0.25, less than or equal to about 0.20, less than or equal to about 0.10, or about 0. In some embodiments, the value of x is greater than or equal to about 0 and less than or equal to about 0.1, greater than or equal to about 0 and less than or equal to about 0.2, greater than or equal to about 0 and less than or equal to about 0.25, greater than or equal to about 0 and less than or equal to about 0.3, greater than or equal to about 0 and less than or equal to about 0.35, greater than or equal to about 0 and less than or equal to about 0.4, greater than or equal to about 0 and less than or equal to about 0.45, greater than or equal to about 0 and less than or equal to about 0.5, greater than or equal to about 0 and less than or equal to about 0.6, greater than or equal to about 0 and less than or equal to about 0.7, greater than or equal to about 0 and less than or equal to about 0.75, greater than or equal to about 0 and less than or equal to about 0.8, greater than or equal to about 0 and less than or equal to about 0.9, greater than or equal to about 0 and less than or equal to about 0.95, greater than or equal to about 0 and less than or equal to about 0.99, greater than or equal to about 0.1 and less than or equal to about 0.2, greater than or equal to about 0.1 and less than or equal to about 0.25, greater than or equal to about 0.1 and less than or equal to about 0.3, greater than or equal to about 0.1 and less than or equal to about 0.35, greater than or equal to about 0.1 and less than or equal to about 0.4, greater than or equal to about 0.1 and less than or equal to about 0.45, greater than or equal to about 0.1 and less than or equal to about 0.5, greater than or equal to about 0.1 and less than or equal to about 0.6, greater than or equal to about 0.1 and less than or equal to about 0.7, greater than or equal to about 0.1 and less than or equal to about 0.75, greater than or equal to about 0.1 and less than or equal to about 0.8, greater than or equal to about 0.1 and less than or equal to about 0.9, greater than or equal to about 0.1 and less than or equal to about 0.95, greater than or equal to about 0.1 and less than or equal to about 0.99, or greater than or equal to about 0.1 and less than or equal to about 1.0. In some embodiments, the value of x is 0.0, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 0.99, or 1.0.

In certain embodiments, “A” is La, “B” is Sr, and “D” is Co. In certain other embodiments, “A” is La, and “D” is Ni. According to some embodiments, non-limiting examples of suitable perovskite compounds include La_{0.8}Sr_{0.2}CoO₃, La_{0.75}Sr_{0.75}CoO₃, La_{0.5}Sr_{0.5}CoO₃, La_{0.25}Sr_{0.75}CoO₃, LaNiO₃, LaFeO₃, SrCoO₃, and Ba_{0.5}Sr_{0.5}Co_{0.08}Fe_{0.2}O₃. In a particular, non-limiting embodiment, the perovskite compound has the formula La₁₋ₓ,(Ba_{0.5}Sr_{0.5})ₓCo_{0.8}Fe_{0.2}O₃. Other combinations of the above designations for “A”, “B,” “D,” and x are also possible.

In some embodiments, the perovskite compound having the formula A₁₋ₓ,BₓDO₃ has a perovskite-type crystal structure. In some such embodiments, the perovskite compound of formula A₁₋ₓBₓDO₃ has a cubic unit cell. According to certain embodiments, the “A” cation(s) may occupy Wyckoff position 1b, ½, ½, ½, the “B” cation(s) may occupy Wyckoff position 1b, ½, ½, ½, the “D” cation(s) may occupy Wyckoff position 1a, 0, 0, 0, and the O²⁻ions may occupy Wyckoff position 3d, ½, 0, 0; 0, ½, 0; 0, 0, ½. In some embodiments, the perovskite compound may have a cubic structure, a rhombohedral structure, an orthorhombic structure, or a monoclinic structure. As illustrative, non-limiting examples, the space group of a perovskite compound having a cubic structure may be Pm-3m, the space group of a perovskite compound having a rhombohedral structure may be R-3c, the space group of a perovskite compound having an orthorhombic structure may be Pnma, and the space group of a perovskite compound having a monoclinic structure may be I2/a. The crystal structure of the perovskite compound may be determined by using powder x-ray diffraction crystallography at a wavelength of 0.15418 nm. It should be noted that perovskite compounds described herein may exhibit variations in crystal structure.

In some embodiments, one or more impurities may be present with the perovskite compound. In certain embodiments, for example, non-perovskite oxides of “A,” “B,” and/or “D” (e.g., oxides having the formula A₂DO₄, B₂DO₄) and/or other compounds comprising “A,” “B,” and/or “D” may be present. Examples of impurities include, but are not limited to, La₂O₃, La₂NiO₄, Co₃O₄, SrO, CoO, and Co metal.

In some embodiments, a perovskite compound catalyzes the hydrogenolysis (e.g., hydrodeoxygenation, hydrodenitrogenation, and/or hydrodesulfurization) of a heteroatom-containing compound. As would be understood by those of ordinary skill in the art, a catalyst generally refers to a compound that increases the rate of a chemical reaction without itself undergoing any permanent change. A hydrogenolysis reaction generally refers to a chemical reaction (e.g., a chemical process in which at least one covalent bond is broken and at least one covalent bond is formed) in which one or more carbon-heteroatom or carbon-carbon bonds of a heteroatom-containing compound are cleaved by hydrogen. In some embodiments, the heteroatom-containing compound comprises nitrogen (N), oxygen (O), and/or sulfur (S). In particular, in some embodiments, the heteroatom-containing compound comprises one or more chemical bonds between carbon and a heteroatom (e.g., N, O, S). The heteroatom-containing compound may be an aromatic compound (e.g., a substituted aromatic compound, a substituted or unsubstituted heteroaromatic compound) or an aliphatic compound (e.g., a substituted aliphatic compound, a substituted or unsubstituted heteroaliphatic compound, a cycloaliphatic compound). In some embodiments, the hydrogenolysis reaction is a hydrodeoxygenation reaction, a hydrodenitrogenation reaction, and/or a hydrodesulfurization reaction. In some embodiments, the perovskite compound catalyzes at least two of the following reactions: a hydrodeoxygenation reaction, a hydrodenitrogenation reaction, and a hydrodesulfurization reaction. In some embodiments, the perovskite compound catalyzes a hydrodeoxygenation reaction, a hydrodenitrogenation reaction, and a hydrodesulfurization reaction.

In some embodiments, the perovskite compound catalyzes the hydrodeoxygenation of an oxygen-containing compound. Hydrodeoxygenation generally refers to a hydrogenolysis reaction in which one or more oxygen atoms are removed from an oxygen-containing compound to produce one or more hydrocarbon products.

The oxygen-containing compound may be an aromatic compound (e.g., a substituted aromatic compound, a substituted or unsubstituted heteroaromatic compound). The aromatic compound may, for example, be a monocyclic, bicyclic, or polycyclic aromatic compound. Non-limiting examples of suitable oxygen-containing aromatic compounds include anisole, guaiacol, veratrole, benzenediol, phenol, cresylic acid, furan, benzofuran, and furfural. In some embodiments, the oxygen-containing compound is an aliphatic compound (e.g., a substituted aliphatic compound, a heteroaliphatic compound, a cycloaliphatic compound). In certain embodiments, the oxygen-containing aliphatic compound comprises an alcohol (e.g., a polyol), a ketone, an aldehyde, a carboxylic acid, an ester, a fatty acid, a fatty acid ester (e.g., a triglyceride), a saccharide (e.g., a monosaccharide, a disaccharide, a polysaccharide), a cellulosic derivative, a lignocellulosic derivative, or any combination thereof.

In some embodiments, hydrodeoxygenation of an oxygen-containing compound results in an at least partially deoxygenated product (e.g., a compound having a lower molecular weight and containing at least one fewer oxygen atom than the oxygen-containing compound). Examples of an at least partially deoxygenated product include, but are not limited to, a substituted aromatic compound (e.g., an aromatic compound comprising at least one oxygen moiety), an alcohol (e.g., a polyol), a ketone, an aldehyde, a carboxylic acid, a fatty acid, a fatty acid ester (e.g., a triglyceride), and a saccharide (e.g., a monosaccharide, a disaccharide, a polysaccharide). In certain embodiments, the at least partially deoxygenated product comprises an aromatic compound having a single oxygen moiety. As used herein, an “oxygen moiety” refers to any oxygen-containing substituent. Non-limiting examples of suitable oxygen moieties include a hydroxyl group, an alkoxy group (e.g., a methoxy group), and a carboxyl group. In certain cases, the at least partially deoxygenated product may be further reacted (e.g., in a hydroprocessing step) to form one or more hydrocarbon products (e.g., one or more compounds consisting of carbon and hydrogen). In some embodiments, hydrodeoxygenation of an oxygen-containing compound results (directly or indirectly) in one or more hydrocarbon products.

In some embodiments, the perovskite compound catalyzes the hydrodenitrogenation of a nitrogen-containing compound. Hydrodenitrogenation generally refers to a hydrogenolysis reaction in which one or more nitrogen atoms are removed from a nitrogen-containing compound to produce one or more hydrocarbon products.

The nitrogen-containing compound may be an aromatic compound (e.g., a substituted aromatic compound, a substituted or unsubstituted heteroaromatic compound). The aromatic compound may, for example, be a monocyclic, bicyclic, or polycyclic aromatic compound. Non-limiting examples of suitable nitrogen-containing aromatic compounds include pyridine, quinolone, isoquinoline, acridine, pyrrole, indole, carbazole, and benzocarbazole. In some embodiments, the nitrogen-containing compound is an aliphatic compound (e.g., a substituted aliphatic compound, a heteroaliphatic compound, a cycloaliphatic compound). In certain embodiments, the nitrogen-containing aliphatic compound comprises an amine and/or an amide.

In some embodiments, hydrodenitrogenation of a nitrogen-containing compound results in an at least partially denitrogenated product (e.g., a compound having a lower molecular weight and containing at least one fewer nitrogen atom than the nitrogen-containing compound). Examples of an at least partially denitrogenated product include, but are not limited to, a substituted aromatic compound (e.g., an aromatic compound comprising at least one nitrogen moiety), an amine, and an amide. In certain embodiments, the at least partially denitrogenated product comprises an aromatic compound having a single nitrogen moiety. As used herein, a “nitrogen moiety” refers to any nitrogen-containing substituent. In certain cases, the at least partially denitrogenated product may be further reacted (e.g., in a hydroproces sing step) to form one or more hydrocarbon products (e.g., one or more compounds consisting of carbon and hydrogen). In some embodiments, hydrodenitrogenation of a nitrogen-containing compound results (directly or indirectly) in one or more hydrocarbon products.

In some embodiments, the perovskite compound catalyzes the hydrodesulfurization of a sulfur-containing compound. Hydrodesulfurization generally refers to a hydrogenolysis reaction in which one or more sulfur atoms are removed from a sulfur-containing compound to produce one or more hydrocarbon products.

The sulfur-containing compound may be an aromatic compound (e.g., a substituted aromatic compound, a substituted or unsubstituted heteroaromatic compound). The aromatic compound may, for example, be a monocyclic, bicyclic, or polycyclic aromatic compound. Non-limiting examples of suitable sulfur-containing aromatic compounds include phenyl mercaptan, thiophene, and benzothiophene. In some embodiments, the sulfur-containing compound is an aliphatic compound (e.g., a substituted aliphatic compound, a heteroaliphatic compound, a cycloaliphatic compound). In certain embodiments, the sulfur-containing aliphatic compound comprises methyl mercaptan, cyclohexylthiol, thiocyclohexane, dimethyl sulfide, dimethylsulfide, or combinations thereof.

In some embodiments, hydrodesulfurization of a sulfur-containing compound results in an at least partially desulfurized product (e.g., a compound having a lower molecular weight and containing at least one fewer sulfur atom than the sulfur-containing compound). A non-limiting example of an at least partially desulfurized product includes a substituted aromatic compound (e.g., an aromatic compound comprising at least one sulfur moiety). In some embodiments, the at least partially desulfurized product comprises an aromatic compound having a single sulfur moiety. As used herein, a “sulfur moiety” refers to any sulfur-containing substituent. A non-limiting example of a suitable sulfur moiety includes a thiol group. In certain cases, the at least partially desulfurized product may be further reacted (e.g., in a hydroproces sing step) to form one or more hydrocarbon products (e.g., one or more compounds consisting of carbon and hydrogen). In some embodiments, hydrodesulfurization of a sulfur-containing compound results (directly or indirectly) in one or more hydrocarbon products.

In some embodiments, the heteroatom-containing compound is a constituent of a first feed stream. The first feed stream may, in some cases, comprise or be derived from crude oil, biomass, and/or a biomass-derived product (e.g., bio-oil). The term “biomass” may refer to material comprising or derived from organic matter, such as plant-based materials. In some embodiments, biomass comprises corn stover, sugarcane bagasse, switchgrass, wood, and/or paper pulp. In certain embodiments, biomass comprises cellulose, hemicellulose, and/or lignin. The term “bio-oil” may refer to a product (e.g., a liquid product) produced through processing of biomass. As an illustrative, non-limiting example, bio-oil may be produced through pyrolysis of biomass.

In some embodiments, the first feed stream has a relatively high concentration of oxygen, nitrogen, and/or sulfur. In some embodiments, the first feed stream has a relatively high oxygen concentration. In certain embodiments, the first feed stream has an oxygen concentration of at least about 0.1 wt %, at least about 0.5 wt %, at least about 1.0 wt %, at least about 1.5 wt %, at least about 2.0 wt %, at least about 5.0 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt %. In certain embodiments, the first feed stream has an oxygen concentration in a range from about 0.1 wt % to about 1.0 wt %, about 0.1 wt % to about 1.5 wt %, about 0.1 wt % to about 2.0 wt %, about 0.1 wt % to about 5.0 wt %, about 0.1 wt % to about 10 wt %, about 0.1 wt % to about 20 wt %, about 0.1 wt % to about 30 wt %, about 0.1 wt % to about 40 wt %, about 0.1 wt % to about 50 wt %, about 0.1 wt % to about 60 wt %, about 0.1 wt % to about 70 wt %, about 0.1 wt % to about 80 wt %, about 1.0 wt % to about 1.5 wt %, about 1.0 wt % to about 2.0 wt %, about 1.0 wt % to about 5.0 wt %, about 1.0 wt % to about 10 wt %, about 1.0 wt % to about 20 wt %, about 1.0 wt % to about 30 wt %, about 1.0 wt % to about 40 wt %, about 1.0 wt % to about 50 wt %, about 1.0 wt % to about 60 wt %, about 1.0 wt % to about 70 wt %, about 1.0 wt % to about 80 wt %, about 5.0 wt % to about 10 wt %, about 5.0 wt % to about 20 wt %, about 5.0 wt % to about 30 wt %, about 5.0 wt % to about 40 wt %, about 5.0 wt % to about 50 wt %, about 5.0 wt % to about 60 wt %, about 5.0 wt % to about 70 wt %, about 5.0 wt % to about 80 wt %, about 10 wt % to about 20 wt %, about 10 wt % to about 30 wt %, about 10 wt % to about 40 wt %, about 10 wt % to about 50 wt %, about 10 wt % to about 60 wt %, about 10 wt % to about 70 wt %, about 10 wt % to about 80 wt %, about 20 wt % to about 30 wt %, about 20 wt % to about 40 wt %, about 20 wt % to about 50 wt %, about 20 wt % to about 60 wt %, about 20 wt % to about 70 wt %, about 20 wt % to about 80 wt %, about 30 wt % to about 40 wt %, about 30 wt % to about 50 wt %, about 30 wt % to about 60 wt %, about 30 wt % to about 70 wt %, about 30 wt % to about 80 wt %, about 40 wt % to about 50 wt %, about 40 wt % to about 60 wt %, about 40 wt % to about 70 wt %, about 40 wt % to about 80 wt %, about 50 wt % to about 60 wt %, about 50 wt % to about 70 wt %, about 50 wt % to about 80 wt %, about 60 wt % to about 70 wt %, about 60 wt % to about 80 wt %, or about 70 wt % to about 80 wt %. The concentration of an element within a feed stream may be obtained using gas chromatography-mass spectrometry (GC-MS).

In some embodiments, the first feed stream has a relatively high nitrogen concentration. In certain embodiments, the first feed stream has a nitrogen concentration of at least about 0.1 wt %, at least about 0.5 wt %, at least about 1.0 wt %, at least about 1.5 wt %, at least about 2.0 wt %, or at least about 5.0 wt %. In certain embodiments, the first feed stream has a nitrogen concentration in a range from about 0.1 wt % to about 1.0 wt %, about 0.1 wt % to about 1.5 wt %, about 0.1 wt % to about 2.0 wt %, about 0.1 wt % to about 5.0 wt %, about 0.5 wt % to about 1.0 wt %, about 0.5 wt % to about 1.5 wt %, about 0.5 wt % to about 2.0 wt %, about 0.5 wt % to about 5.0 wt %, about 1.0 wt % to about 1.5 wt %, about 1.0 wt % to about 2.0 wt %, about 1.0 wt % to about 5.0 wt %, or about 2.0 wt % to about 5.0 wt %.

In some embodiments, the first feed stream has a relatively high sulfur concentration. In certain embodiments, the first feed stream has a sulfur concentration of at least about 0.1 wt %, at least about 0.5 wt %, at least about 1.0 wt %, at least about 1.5 wt %, at least about 2.0 wt %, at least about 5.0 wt %, or at least about 10 wt %. In certain embodiments, the first feed stream has a sulfur concentration in a range from about 0.1 wt % to about 1.0 wt %, about 0.1 wt % to about 1.5 wt %, about 0.1 wt % to about 2.0 wt %, about 0.1 wt % to about 5.0 wt %, about 0.1 wt % to about 10 wt %, about 0.5 wt % to about 1.0 wt %, about 0.5 wt % to about 1.5 wt %, about 0.5 wt % to about 2.0 wt %, about 0.5 wt % to about 5.0 wt %, about 0.5 wt % to about 10 wt %, about 1.0 wt % to about 1.5 wt %, about 1.0 wt % to about 2.0 wt %, about 1.0 wt % to about 5.0 wt %, about 1.0 wt % to about 10 wt %, about 2.0 wt % to about 5.0 wt %, about 2.0 wt % to about 10 wt %, or about 5.0 wt % to about 10 wt %.

In some embodiments, the one or more hydrocarbon products (e.g., one or more compounds consisting of carbon and hydrogen) formed by perovskite-catalyzed hydrogenolysis (e.g., hydrodeoxygenation, hydrodenitrogenation, and/or hydrodesulfurization) comprise an aromatic hydrocarbon and/or an aliphatic hydrocarbon. The aromatic hydrocarbon may be an unsubstituted aromatic hydrocarbon (e.g., benzene) or a substituted aromatic hydrocarbon (e.g., toluene). The aliphatic hydrocarbon may be saturated or unsaturated and may be substituted or unsubstituted. According to certain embodiments, the aliphatic hydrocarbon is an alkane (e.g., methane, ethane, propane, butane, pentane, hexane). In certain cases, the alkane is a cycloalkane (e.g., cyclopentane, cyclohexane). In some embodiments, the one or more hydrocarbon products are constituents of one or more product streams.

In some embodiments, the one or more hydrocarbon products comprise an aromatic hydrocarbon. The yield of the aromatic hydrocarbon may be relatively high. In certain embodiments, the yield of the aromatic hydrocarbon is at least about 50 carbon-mol %, at least about 60 carbon-mol %, at least about 70 carbon-mol %, at least about 80 carbon-mol %, at least about 90 carbon-mol %, at least about 95 carbon-mol %, at least about 99 carbon-mol %, or about 100 carbon-mol %. In certain embodiments, the yield of the aromatic hydrocarbon is in a range from about 50 carbon-mol % to about 90 carbon-mol %, about 50 carbon-mol % to about 95 carbon-mol %, about 50 carbon-mol % to about 99 carbon-mol %, about 50 carbon-mol % to about 100 carbon-mol %, about 60 carbon-mol % to about 90 carbon-mol %, about 60 carbon-mol % to about 95 carbon-mol %, about 60 carbon-mol % to about 99 carbon-mol %, about 60 carbon-mol % to about 100 carbon-mol %, about 70 carbon-mol % to about 90 carbon-mol %, about 70 carbon-mol % to about 95 carbon-mol %, about 70 carbon-mol % to about 99 carbon-mol %, about 70 carbon-mol % to about 100 carbon-mol %, about 80 carbon-mol % to about 90 carbon-mol %, about 80 carbon-mol % to about 95 carbon-mol %, about 80 carbon-mol % to about 99 carbon-mol %, about 80 carbon-mol % to about 100 carbon-mol %, about 90 carbon-mol % to about 95 carbon-mol %, about 90 carbon-mol % to about 99 carbon-mol %, about 90 carbon-mol % to about 100 carbon-mol %, or about 95 carbon-mol % to about 100 carbon-mol %. The yield of the aromatic hydrocarbon may be determined by evaluating the one or more hydrocarbon products via GC-MS. In particular, GC-MS may be used to determine the number of moles of a product species (e.g., an aromatic hydrocarbon). The number of moles of the product species may then be converted to the number of moles of carbon, and the yield may be calculated by dividing the number of moles of carbon in the product species by the number of moles of carbon in the heteroatom-containing compound.

In some embodiments, the total yield of aromatic hydrocarbons is relatively high. In certain embodiments, the total yield of aromatic hydrocarbons is at least about 50 carbon-mol %, at least about 60 carbon-mol %, at least about 70 carbon-mol %, at least about 80 carbon-mol %, at least about 90 carbon-mol %, at least about 95 carbon-mol %, at least about 99 carbon-mol %, or about 100 carbon-mol %. In some embodiments, the total yield of aromatic hydrocarbons is in a range from about 50 carbon-mol % to about 90 carbon-mol %, about 50 carbon-mol % to about 95 carbon-mol %, about 50 carbon-mol % to about 99 carbon-mol %, about 50 carbon-mol % to about 100 carbon-mol %, about 60 carbon-mol % to about 90 carbon-mol %, about 60 carbon-mol % to about 95 carbon-mol %, about 60 carbon-mol % to about 99 carbon-mol %, about 60 carbon-mol % to about 100 carbon-mol %, about 70 carbon-mol % to about 90 carbon-mol %, about 70 carbon-mol % to about 95 carbon-mol %, about 70 carbon-mol % to about 99 carbon-mol %, about 70 carbon-mol % to about 100 carbon-mol %, about 80 carbon-mol % to about 90 carbon-mol %, about 80 carbon-mol % to about 95 carbon-mol %, about 80 carbon-mol % to about 99 carbon-mol %, about 80 carbon-mol % to about 100 carbon-mol %, about 90 carbon-mol % to about 95 carbon-mol %, about 90 carbon-mol % to about 99 carbon-mol %, about 90 carbon-mol % to about 100 carbon-mol %, or about 95 carbon-mol % to about 100 carbon-mol %. The total yield of aromatic hydrocarbons may be obtained by adding the yields of all aromatic hydrocarbon product species.

In some embodiments, the one or more hydrocarbon products comprise an aliphatic hydrocarbon (e.g., an alkane). The yield of the aliphatic hydrocarbon may be relatively high. In some embodiments, the yield of the aliphatic hydrocarbon is at least about 50 carbon-mol %, at least about 60 carbon-mol %, at least about 70 carbon-mol %, at least about 80 carbon-mol %, at least about 90 carbon-mol %, at least about 95 carbon-mol %, at least about 99 carbon-mol %, or about 100 carbon-mol %. In some embodiments, the yield of the aliphatic hydrocarbon is in a range from about 50 carbon-mol % to about 90 carbon-mol %, about 50 carbon-mol % to about 95 carbon-mol %, about 50 carbon-mol % to about 99 carbon-mol %, about 50 carbon-mol % to about 100 carbon-mol %, about 60 carbon-mol % to about 90 carbon-mol %, about 60 carbon-mol % to about 95 carbon-mol %, about 60 carbon-mol % to about 99 carbon-mol %, about 60 carbon-mol % to about 100 carbon-mol %, about 70 carbon-mol % to about 90 carbon-mol %, about 70 carbon-mol % to about 95 carbon-mol %, about 70 carbon-mol % to about 99 carbon-mol %, about 70 carbon-mol % to about 100 carbon-mol %, about 80 carbon-mol % to about 90 carbon-mol %, about 80 carbon-mol % to about 95 carbon-mol %, about 80 carbon-mol % to about 99 carbon-mol %, about 80 carbon-mol % to about 100 carbon-mol %, about 90 carbon-mol % to about 95 carbon-mol %, about 90 carbon-mol % to about 99 carbon-mol %, about 90 carbon-mol % to about 100 carbon-mol %, or about 95 carbon-mol % to about 100 carbon-mol %.

In some embodiments, the total yield of aliphatic hydrocarbons (e.g., alkanes) is relatively high. In some embodiments, the total yield of aliphatic hydrocarbons is at least about 50 carbon-mol %, at least about 60 carbon-mol %, at least about 70 carbon-mol %, at least about 80 carbon-mol %, at least about 90 carbon-mol %, at least about 95 carbon-mol %, at least about 99 carbon-mol %, or about 100 carbon-mol %. In some embodiments, the total yield of aliphatic hydrocarbons is in a range from about 50 carbon-mol % to about 90 carbon-mol %, about 50 carbon-mol % to about 95 carbon-mol %, about 50 carbon-mol % to about 99 carbon-mol %, about 50 carbon-mol % to about 100 carbon-mol %, about 60 carbon-mol % to about 90 carbon-mol %, about 60 carbon-mol % to about 95 carbon-mol %, about 60 carbon-mol % to about 99 carbon-mol %, about 60 carbon-mol % to about 100 carbon-mol %, about 70 carbon-mol % to about 90 carbon-mol %, about 70 carbon-mol % to about 95 carbon-mol %, about 70 carbon-mol % to about 99 carbon-mol %, about 70 carbon-mol % to about 100 carbon-mol %, about 80 carbon-mol % to about 90 carbon-mol %, about 80 carbon-mol % to about 95 carbon-mol %, about 80 carbon-mol % to about 99 carbon-mol %, about 80 carbon-mol % to about 100 carbon-mol %, about 90 carbon-mol % to about 95 carbon-mol %, about 90 carbon-mol % to about 99 carbon-mol %, about 90 carbon-mol % to about 100 carbon-mol %, or about 95 carbon-mol % to about 100 carbon-mol %.

In some embodiments, the one or more hydrocarbon products comprise a cycloaliphatic hydrocarbon. The yield of the cycloaliphatic hydrocarbon may be relatively high. In some embodiments, the yield of the cycloaliphatic hydrocarbon is at least about 50 carbon-mol %, at least about 60 carbon-mol %, at least about 70 carbon-mol %, at least about 80 carbon-mol %, at least about 90 carbon-mol %, at least about 95 carbon-mol %, at least about 99 carbon-mol %, or about 100 carbon-mol %. In some embodiments, the yield of the cycloaliphatic hydrocarbon is in a range from about 50 carbon-mol % to about 90 carbon-mol %, about 50 carbon-mol % to about 95 carbon-mol %, about 50 carbon-mol % to about 99 carbon-mol %, about 50 carbon-mol % to about 100 carbon-mol %, about 60 carbon-mol % to about 90 carbon-mol %, about 60 carbon-mol % to about 95 carbon-mol %, about 60 carbon-mol % to about 99 carbon-mol %, about 60 carbon-mol % to about 100 carbon-mol %, about 70 carbon-mol % to about 90 carbon-mol %, about 70 carbon-mol % to about 95 carbon-mol %, about 70 carbon-mol % to about 99 carbon-mol %, about 70 carbon-mol % to about 100 carbon-mol %, about 80 carbon-mol % to about 90 carbon-mol %, about 80 carbon-mol % to about 95 carbon-mol %, about 80 carbon-mol % to about 99 carbon-mol %, about 80 carbon-mol % to about 100 carbon-mol %, about 90 carbon-mol % to about 95 carbon-mol %, about 90 carbon-mol % to about 99 carbon-mol %, about 90 carbon-mol % to about 100 carbon-mol %, or about 95 carbon-mol % to about 100 carbon-mol %.

In some embodiments, the total yield of cycloaliphatic hydrocarbons is relatively high. In some embodiments, the total yield of cycloaliphatic hydrocarbons is at least about 50 carbon-mol %, at least about 60 carbon-mol %, at least about 70 carbon-mol %, at least about 80 carbon-mol %, at least about 90 carbon-mol %, at least about 95 carbon-mol %, at least about 99 carbon-mol %, or about 100 carbon-mol %. In some embodiments, the total yield of cycloaliphatic hydrocarbons is in a range from about 50 carbon-mol % to about 90 carbon-mol %, about 50 carbon-mol % to about 95 carbon-mol %, about 50 carbon-mol % to about 99 carbon-mol %, about 50 carbon-mol % to about 100 carbon-mol %, about 60 carbon-mol % to about 90 carbon-mol %, about 60 carbon-mol % to about 95 carbon-mol %, about 60 carbon-mol % to about 99 carbon-mol %, about 60 carbon-mol % to about 100 carbon-mol %, about 70 carbon-mol % to about 90 carbon-mol %, about 70 carbon-mol % to about 95 carbon-mol %, about 70 carbon-mol % to about 99 carbon-mol %, about 70 carbon-mol % to about 100 carbon-mol %, about 80 carbon-mol % to about 90 carbon-mol %, about 80 carbon-mol % to about 95 carbon-mol %, about 80 carbon-mol % to about 99 carbon-mol %, about 80 carbon-mol % to about 100 carbon-mol %, about 90 carbon-mol % to about 95 carbon-mol %, about 90 carbon-mol % to about 99 carbon-mol %, about 90 carbon-mol % to about 100 carbon-mol %, or about 95 carbon-mol % to about 100 carbon-mol %.

Certain embodiments described herein are related to systems. According to some embodiments, the system comprises a first feed stream comprising a heteroatom-containing compound (e.g., any heteroatom-containing compound described above or elsewhere). In some embodiments, the first feed stream comprises two or more streams, each stream comprising a heteroatom-containing compound. In certain embodiments, the two or more streams of the first feed stream comprise different heteroatom-containing compounds (e.g., a first stream comprises a first heteroatom-containing compound and a second stream comprises a second heteroatom-containing compound). The different heteroatom-containing compounds may comprise the same or different heteroatoms. In certain embodiments, for example, a first heteroatom-containing compound of a first stream and a second heteroatom-containing compound of a second stream comprise one or more different heteroatoms. In certain other embodiments, a first heteroatom-containing compound of a first stream and a second heteroatom-containing compound of a second stream comprise the same heteroatom(s) (e.g., O, N, and/or S). The first feed stream, and any component streams, may be in a liquid phase, a vapor phase, or a mixed vapor/liquid phase.

In some embodiments, the system further comprises a second feed stream comprising hydrogen gas (H₂).

In some embodiments, the system further comprises a perovskite compound (e.g., any perovskite compound described above or elsewhere). According to certain embodiments, the system comprises a reactor. The reactor may, in some cases, comprise a catalyst bed comprising the perovskite compound. In certain embodiments, the catalyst bed may comprise two or more perovskite compounds (e.g., a first perovskite compound and a second, different perovskite compound). In certain embodiments, the catalyst bed comprises one or more perovskite compounds (e.g., compounds having the formula A₁₋ₓ,BₓDO₃) and one or more non-perovskite catalysts (e.g., non-perovskite hydrogenolysis catalysts). In some embodiments, the one or more non-perovskite catalysts (e.g., non-perovskite hydrogenolysis catalysts) comprise molybdenum (Mo), tungsten (W), cobalt (Co), and/or platinum (Pt). In some embodiments, the catalyst bed comprises one or more perovskite compounds and does not comprise any non-perovskite catalysts. In some embodiments, the catalyst bed comprises one or more perovskite compounds and does not comprise any non-perovskite hydrogenolysis catalysts.

The reactor may be any suitable reactor in which a chemical reaction can occur. Examples of suitable types of reactors include, but are not limited to, fixed bed reactors, fluid bed reactors, tubular reactors, and continuous stirred tank reactors. The reactor may be operated under batch, semi-batch, or continuous flow conditions. In some embodiments, the reactor is a down-flow reactor (e.g., a reactor in which one or more feed streams flow downward through the catalyst bed).

The reactor may comprise a housing having any suitable size and shape. In some embodiments, the reactor housing has a substantially parallelepiped shape, a substantially rectangular prismatic shape, a substantially cylindrical shape, and/or a substantially pyramidal shape. In some embodiments, the reactor housing has an outer diameter of at least about 0.5 cm, at least about 1 cm, at least about 5 cm, at least about 10 cm, at least about 20 cm, at least about 50 cm, at least about 100 cm, at least about 200 cm, or at least about 500 cm. In some embodiments, the reactor housing has an outer diameter in a range from about 0.5 cm to about 1 cm, about 0.5 cm to about 5 cm, about 0.5 cm to about 10 cm, about 0.5 cm to about 20 cm, about 0.5 cm to about 50 cm, about 0.5 cm to about 100 cm, about 0.5 cm to about 200 cm, about 0.5 cm to about 500 cm, about 1 cm to about 5 cm, about 1 cm to about 10 cm, about 1 cm to about 20 cm, about 1 cm to about 50 cm, about 1 cm to about 100 cm, about 1 cm to about 200 cm, about 1 cm to about 500 cm, about 10 cm to about 50 cm, about 10 cm to about 100 cm, about 10 cm to about 200 cm, about 10 cm to about 500 cm, about 50 cm to about 100 cm, about 50 cm to about 200 cm, about 50 cm to about 500 cm, about 100 cm to about 200 cm, or about 100 cm to about 500 cm. The reactor housing may be formed of any suitable material. Non-limiting examples of suitable materials include stainless steel, aluminum, and plastic.

As described above, the reactor may comprise a catalyst bed comprising a perovskite compound. The perovskite compound may be in any suitable form. In some embodiments, the catalyst bed comprises pellets and/or beads of the perovskite compound. The pellets and/or beads of the perovskite compound may have any suitable size and shape. In some embodiments, for example, the pellets and/or beads of the perovskite compound are substantially cylindrical, substantially spherical, substantially elliptical, substantially rectangular, irregularly shaped, or any other shape. In certain embodiments, the perovskite compound is in powder form.

In some embodiments, the perovskite compound is mixed with an inert diluent prior to forming the catalyst bed. Non-limiting examples of suitable diluents include α-Al₂O₃ and silicon carbide (SiC).

In some embodiments, the catalyst bed comprises the perovskite compound positioned on a support. Any suitable support may be used. Non-limiting examples of suitable materials for the support include carbon, metal oxides (e.g., alumina), silica, and potassium oxide. In some embodiments, the support may comprise a monolith (e.g., a ceramic monolith). In some embodiments, the catalyst bed comprises the perovskite compound and/or a non-perovskite catalyst (e.g., a non-perovskite hydrogenolysis catalyst) positioned on a perovskite support.

In some embodiments, the perovskite compound catalyzes hydrogenolysis (e.g., hydrodeoxygenation, hydrodenitrogenation, hydrodesulfurization) of the heteroatom-containing compound to produce one or more hydrocarbon products. In some embodiments, the system further comprises one or more product streams comprising one or more hydrocarbon compounds.

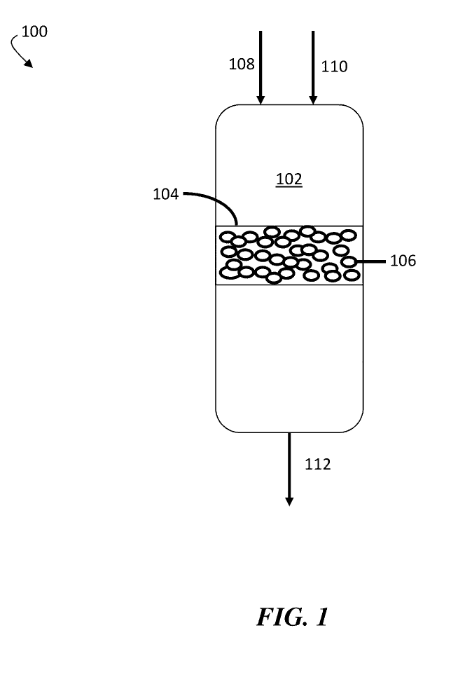

A schematic diagram of an exemplary system is illustrated in FIG. 1. In FIG. 1, system 100 comprises reactor 102. In the non-limiting, illustrative embodiment shown in FIG. 1, reactor 102 comprises catalyst bed 104, which comprises pellets 106 of a perovskite compound. System 100 further comprises, according to some embodiments, first feed stream 108 comprising a heteroatom-containing compound. In addition, system 100 comprises second feed stream 110 comprising hydrogen gas. System 100 also comprises product stream 112 comprising one or more hydrocarbon products.

In operation, first feed stream 108 comprising the heteroatom-containing compound and second feed stream 110 comprising hydrogen gas are introduced into reactor 102 through one or more fluid inlets. In some embodiments, first feed stream 108 and second feed stream 110 are mixed to form a mixed feed stream (not shown in FIG. 1), which flows downward through catalyst bed 106. As the mixed feed stream comprising the heteroatom-containing compound and hydrogen gas directly contacts catalyst bed 104, pellets 106 of the perovskite compound catalyze the hydrogenolysis (e.g., hydrodeoxygenation, hydrodenitrogenation, hydrodesulfurization) of the heteroatom-containing compound to produce product stream 112 comprising one or more hydrocarbon products. Product stream 112 then exits reactor 102 through a fluid outlet.

Certain aspects are related to methods of catalytic hydrogenolysis (e.g., hydrodeoxygenation, hydrodenitrogenation, and/or hydrodesulfurization). In some embodiments, the method comprises contacting a perovskite compound with a heteroatom-containing compound in the presence of hydrogen gas (H₂). In some embodiments, the perovskite compound catalyzes hydrogenolysis of the heteroatom-containing compound to produce one or more hydrocarbon products. The perovskite compound, heteroatom-containing compound, and one or more hydrocarbon products may be any of the compounds described above or elsewhere.

According to certain embodiments, the method comprises introducing a first feed stream comprising a heteroatom-containing compound into a reactor comprising a catalyst bed comprising a perovskite compound. In some embodiments, the first feed stream comprises two or more streams, each stream comprising a heteroatom-containing compound. In certain embodiments, the two or more streams of the first feed stream comprise different heteroatom-containing compounds (e.g., a first stream comprises a first heteroatom-containing compound and a second stream comprises a second heteroatom-containing compound). The different heteroatom-containing compounds may comprise the same or different heteroatoms. In certain embodiments, for example, a first heteroatom-containing compound of a first stream and a second heteroatom-containing compound of a second stream comprise one or more different heteroatoms. In certain other embodiments, a first heteroatom-containing compound of a first stream and a second heteroatom-containing compound of a second stream comprise the same heteroatom(s) (e.g., O, N, and/or S). The first feed stream, and any component streams, may be in a liquid phase, a vapor phase, or a mixed vapor/liquid phase. In certain embodiments, two or more streams of the first feed stream may be co-fed into the reactor.

In some embodiments, the pressure of the first feed stream comprising the heteroatom-containing compound is relatively low. In certain embodiments, the pressure of the first feed stream is about 50 atm or less, about 40 atm or less, about 30 atm or less, about 20 atm or less, about 10 atm or less, about 5 atm or less, about 2 atm or less, about 1 atm or less, about 0.5 atm or less, about 0.1 atm or less, about 0.05 atm or less, about 0.01 atm or less, about 0.005 atm or less, about 0.001 atm or less, about 0.005 atm or less, or about 0.0001 atm or less. In some embodiments, the pressure of the first feed stream is in a range of about 0.0001 atm to about 0.001 atm, about 0.0001 atm to about 0.01 atm, about 0.0001 atm to about 0.1 atm, about 0.0001 atm to about 1 atm, about 0.0001 atm to about 5 atm, about 0.0001 atm to about 10 atm, about 0.0001 atm to about 20 atm, about 0.0001 atm to about 30 atm, about 0.0001 atm to about 40 atm, or about 0.0001 atm to about 50 atm.

In some embodiments, the method further comprises introducing a second feed stream comprising hydrogen gas (H₂) into the reactor. According to some embodiments, the catalytic hydrogenolysis reaction is conducted under a relatively low hydrogen gas pressure. In some embodiments, the pressure of the second feed stream is about 50 atm or less, about 40 atm or less, about 30 atm or less, about 20 atm or less, about 10 atm or less, about 5 atm or less, about 2 atm or less, about 1 atm or less, about 0.5 atm or less, about 0.2 atm or less, or about 0.1 atm or less. In some embodiments, the pressure of the second feed stream is in a range from about 0.1 atm to about 0.5 atm, about 0.1 atm to about 1 atm, about 0.1 atm to about 2 atm, about 0.1 atm to about 5 atm, about 0.1 atm to about 10 atm, about 0.1 atm to about 20 atm, about 0.1 atm to about 30 atm, about 0.1 atm to about 40 atm, about 0.1 atm to about 50 atm, about 0.5 atm to about 1 atm, about 0.5 atm to about 2 atm, about 0.5 atm to about 5 atm, about 0.5 atm to about 10 atm, about 0.5 atm to about 20 atm, about 0.5 atm to about 30 atm, about 0.5 atm to about 40 atm, about 0.5 atm to about 50 atm, about 1 atm to about 2 atm, about 1 atm to about 5 atm, about 1 atm to about 10 atm, about 1 atm to about 20 atm, about 1 atm to about 30 atm, about 1 atm to about 40 atm, about 1 atm to about 50 atm, about 10 atm to about 20 atm, about 10 atm to about 30 atm, about 10 atm to about 40 atm, or about 10 atm to about 50 atm.

In some embodiments, the method comprises flowing the first feed stream and the second feed stream through the reactor such that the first feed stream and the second feed stream directly contact the catalyst bed. In certain embodiments, the first feed stream and/or the second feed stream flow through the catalyst bed. In certain embodiments, the first feed stream and/or the second feed stream flow over a surface of the catalyst bed. The first feed stream and/or the second feed stream may flow through the reactor at any suitable flow rate or weight hourly space velocity.

In certain embodiments, the first feed stream and the second feed stream are mixed to form a mixed feed stream. In some cases, the mixed feed stream may be formed prior to the first feed stream and/or the second feed stream coming into direct contact with the catalyst bed. In some embodiments, the method comprises flowing the mixed feed stream through the reactor such that the mixed feed stream directly contacts the catalyst bed. In certain embodiments, the mixed feed stream flows through the catalyst bed. In certain embodiments, the mixed feed stream flows over a surface of the catalyst bed. The mixed feed stream may flow through the reactor at any suitable flow rate or weight hourly space velocity.

In some embodiments, the perovskite compound of the catalyst bed catalyzes a hydrogenolysis reaction (e.g., a hydrodeoxygenation reaction, a hydrodenitrogenation reaction, and/or a hydrodesulfurization reaction) to produce one or more product streams comprising one or more hydrocarbon compounds. In some embodiments, the method is performed in a petroleum refinery, a natural gas processing refinery, or a biomass processing plant.

Those of ordinary skill in the art will be able to determine suitable conditions under which to contact a perovskite compound and a heteroatom-containing compound. Conditions which may be varied include, but are not limited to, temperature, pressure, and time of exposure.

In some embodiments, the method of catalytic hydrogenolysis (e.g., hydrodenitrogenation, hydrodeoxygenation, and/or hydrodesulfurization) is conducted at a relatively low temperature. In certain embodiments, the method of catalytic hydrogenolysis is conducted at a temperature of about 400° C. or less, about 350° C. or less, about 325° C. or less, about 300° C. or less, about 250° C. or less, about 200° C. or less, about 150° C. or less, or about 100° C. or less. In some embodiments, the method of catalytic hydrogenolysis is conducted at a temperature in a range of about 100° C. to about 200° C., about 100° C. to about 250° C., about 100° C. to about 300° C., about 100° C. to about 325° C., about 100° C. to about 350° C., or about 100° C. to about 400° C.

In some embodiments, the method of catalytic hydrogenolysis (e.g., hydrodenitrogenation, hydrodeoxygenation, hydrodesulfurization) is conducted at a relatively low total pressure. In certain embodiments, the catalytic hydrogenolysis reaction is conducted at a total pressure of about 50 atm or less, about 40 atm or less, about 30 atm or less, about 20 atm or less, about 10 atm or less, about 5 atm or less, about 2 atm or less, about 1 atm or less, or about 0.5 atm or less. In certain embodiments, the catalytic hydrogenolysis reaction is conducted at a total pressure in a range from about 0.5 atm to about 1 atm, about 0.5 atm to about 2 atm, about 0.5 atm to about 5 atm, about 0.5 atm to about 10 atm, about 0.5 atm to about 20 atm, about 0.5 atm to about 30 atm, about 0.5 atm to about 40 atm, about 0.5 atm to about 50 atm, about 1 atm to about 2 atm, about 1 atm to about 5 atm, about 1 atm to about 10 atm, about 1 atm to about 20 atm, about 1 atm to about 30 atm, about 1 atm to about 40 atm, about 1 atm to about 50 atm, about 10 atm to about 20 atm, about 10 atm to about 30 atm, about 10 atm to about 40 atm, or about 10 atm to about 50 atm.

As used herein, the term “hydrocarbon” includes organic compounds consisting of hydrogen and carbon. A hydrocarbon may be saturated or unsaturated (at one or more locations) and may have a linear, branched, monocyclic, or polycyclic structure. A hydrocarbon may be aliphatic or aromatic, and may contain one or more functional groups (e.g., alkyl, alkenyl, and/or alkynyl functional groups).

As used herein, the term “aliphatic” includes both saturated and unsaturated, non-aromatic, straight-chain (i.e., unbranched), branched, acyclic, and cyclic (i.e., carbocylic) hydrocarbons, which are optionally substituted with one or more functional groups. As will be appreciated by one of ordinary skill in the art, “aliphatic” groups include, but are not limited to, alkyl, alkenyl, alkynyl, cycloalkyl, cycloalkenyl, and cycloalkynyl moieties. In certain embodiments, as used herein, “aliphatic” is used to indicate those aliphatic groups or compounds (cyclic, acyclic, substituted, unsubstituted, branched, or unbranched) having 1-20 carbon atoms. Examples of aliphatic hydrocarbon compounds include, but are not limited to, methane, ethane, propane, butane, isobutane, pentane, methylbutane, dimethylpropane, hexane, heptane, octane, isooctane, nonane, decane, ethene, propene, butene, methylpropene, pentene, methylbutene, hexene, octene, trimethylpentene, ethyne, propyne, butyne, pentyne, hexyne, heptyne, and octyne. Substituents of aliphatic groups or compounds include, but are not limited to, any of the substituents described herein, that result in the formation of a stable moiety or compound (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “alkyl” is given its ordinary meaning in the art and refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In some cases, the alkyl group may be a lower alkyl group, i.e., an alkyl group having 1 to 10 carbon atoms (e.g., methyl, ethyl, propyl, butyl, pentyl, hexyl, heptyl, octyl, nonyl, or decyl). In some embodiments, a straight-chain or branched-chain alkyl may have 30 or fewer carbon atoms in its backbone, and, in some cases, 20 or fewer. In some embodiments, a straight-chain or branched-chain alkyl may have 12 or fewer carbon atoms in its backbone (e.g., C₁-C₁₂ for straight chain, C₃-C₁₂ for branched chain), 6 or fewer, or 4 or fewer. Likewise, cycloalkyls may have from 3-10 carbon atoms in their ring structure, or 5, 6, or 7 carbons in the ring structure. Examples of alkyl groups include, but are not limited to, methyl, ethyl, propyl, isopropyl, cyclopropyl, butyl, isobutyl, t-butyl, cyclobutyl, hexyl, and cyclohexyl.

The term “alkylene” as used herein refers to a bivalent alkyl group. An “alkylene” group is a polymethylene group, i.e., —(CH₂)_{z}—, wherein z is a positive integer, e.g., from 1 to 20, from 1 to 10, from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described herein for a substituted aliphatic group.

Generally, the suffix “-ene” is used to describe a bivalent group. Thus, any of the terms defined herein can be modified with the suffix “-ene” to describe a bivalent version of that moiety. For example, a bivalent carbocycle is “carbocyclylene,” a bivalent aryl ring is “arylene,” a bivalent benzene ring is “phenylene,” a bivalent heterocycle is “heterocyclylene,” a bivalent heteroaryl ring is “heteroarylene,” a bivalent alkyl chain is “alkylene,” a bivalent alkenyl chain is “alkenylene,” a bivalent alkynyl chain is “alkynylene,” a bivalent heteroalkyl chain is “heteroalkylene,” a bivalent heteroalkenyl chain is “heteroalkenylene,” a bivalent heteroalkynyl chain is “heteroalkynylene,” and so forth.

The terms “alkenyl” and “alkynyl” are given their ordinary meaning in the art and refer to unsaturated aliphatic groups analogous in length and possible substitution to the alkyl groups described above, but that contain at least one double or triple bond respectively.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, allyl, n-butyl, sec-butyl, isobutyl, t-butyl, n-pentyl, sec-pentyl, isopentyl, t-pentyl, n-hexyl, sec-hexyl, moieties and the like, which again, may bear one or more substituents. Alkenyl groups include, but are not limited to, for example, ethenyl, propenyl, butenyl, 1-methyl-2-buten-l-yl, and the like. Representative alkynyl groups include, but are not limited to, ethynyl, 2-propynyl (propargyl), 1-propynyl and the like.

The term “heteroaliphatic,” as used herein, refers to an aliphatic moiety or compound, as defined herein, which includes both saturated and unsaturated, non-aromatic, straight-chain (i.e., unbranched), branched, acyclic, cyclic (i.e., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties or compounds are substituted by independent replacement of one or more of the hydrogen atoms thereon with one or more substituents. As will be appreciated by one of ordinary skill in the art, “heteroaliphatic” groups include, but are not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkane,” “heteroalkene,” “heteroalkyne,” “heteroalkyl,” “heteroalkenyl,” “heteroalkynyl,” and the like. Furthermore, as used herein, the terms “heteroalkane,” “heteroalkene,” “heteroalkyne,” “heteroalkyl,” “heteroalkenyl,” “heteroalkynyl”, and the like encompass both substituted and unsubstituted groups and compounds. In certain embodiments, as used herein, “heteroaliphatic” is used to indicate those heteroaliphatic groups and compounds (cyclic, acyclic, substituted, unsubstituted, branched or unbranched) having 1-20 carbon atoms.

Substituents of heteroaliphatic groups or compounds include, but are not limited to, any of the substituents described herein, that result in the formation of a stable moiety or compound (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “cycloaliphatic,” as used herein, refers to saturated or partially unsaturated cyclic aliphatic monocyclic, bicyclic, or polycyclic ring systems, as described herein, having from 3 to 14 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The term “cycloaliphatic” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, that has a single point of attachment to the rest of the molecule, or a C₉-C₁₆ tricyclic hydrocarbon that is completely saturated or that contains one or more units of unsaturation, but which is not aromatic, that has a single point of attachment to the rest of the molecule. Examples of cycloaliphatic hydrocarbons include, but are not limited to, cyclopropane, cyclobutane, cyclopentane, cyclohexane, cycloheptane, cyclooctane, cyclononane, cyclodecane, cyclopropene, cyclobutene, methylcyclopropene, cyclopentene, cyclohexene, cycloheptene, cyclooctene, cyclononene, cyclodecene, cyclooctyne, cyclononyne, and cyclodecyne.

As used herein, the term “alkane” refers to a saturated hydrocarbon molecule. For certain implementations, an alkane can include from 1 to 100 carbon atoms. The term “lower alkane” refers to an alkane that includes from 1 to 20 carbon atoms, such as from 1 to 10 carbon atoms, while the term “upper alkane” refers to an alkane that includes more than 20 carbon atoms, such as from 21 to 100 carbon atoms. The term “branched alkane” refers to an alkane that includes a set of branches, while the term “unbranched alkane” refers to an alkane that is straight-chained. The term “cycloalkane” refers to an alkane that includes a set of ring structures, such as a single ring structure or a bicyclo or higher order cyclic structure. The term “heteroalkane” refers to an alkane that has one or more carbon atoms replaced by one or more heteroatoms, such as N, Si, S, O, and P. The term “substituted alkane” refers to an alkane that has a set of its hydrogen atoms replaced by a set of substituent groups, while the term “unsubstituted alkane” refers to an alkane that lacks such replacement. Combinations of the above terms can be used to refer to an alkane having a combination of characteristics. For example, the term “branched lower alkane” can be used to refer to an alkane that includes from 1 to 20 carbon atoms and a set of branches.

The terms “aromatic” or “aryl” are given their ordinary meaning in the art and refer to aromatic carbocyclic groups or compounds, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aromatic compound and/or aryl group may be optionally substituted, as described herein. Substituents include, but are not limited to, any of the previously mentioned substituents, i.e., the substituents recited for aliphatic groups or compounds, or for other groups or compounds as disclosed herein. In some cases, an aryl group is a stable mono- or polycyclic unsaturated moiety having preferably 3-14 carbon atoms, each of which may be substituted or unsubstituted. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups. Examples of aromatic compounds include, but are not limited to, benzene, toluene, ethylbenzene, propylbenzene, cumene, isobutylbenzene, dodecylbenzene, styrene, xylene, cymene, mesitylene, naphthalene, biphenyl, and anthracene.

As used herein, the term “heteroaromatic” or “heteroaryl” means a monocyclic or polycyclic heteroaromatic ring (or radical thereof) comprising carbon atom ring members and one or more heteroatom ring members (such as, for example, oxygen, sulfur or nitrogen). Typically, the heteroaromatic ring has from 5 to about 8 ring members in which at least 1 ring member is a heteroatom selected from oxygen, sulfur, and nitrogen. In another embodiment, the heteroaromatic ring is a 5- or 6-membered ring and may contain from 1 to about 4 heteroatoms. In another embodiment, the heteroaromatic ring system has 7 to 8 ring members and may contain from 1 to about 6 heteroatoms. Representative heteroaryl groups include pyridyl, furyl, thienyl, pyrrolyl, oxazolyl, imidazolyl, indolizinyl, thiazolyl, isoxazolyl, pyrazolyl, isothiazolyl, pyridazinyl, pyrimidinyl, pyrazinyl, triazinyl, triazolyl, pyridinyl, thiadiazolyl, pyrazinyl, quinolyl, isoquniolyl, indazolyl, benzoxazolyl, benzofuryl, benzothiazolyl, indolizinyl, imidazopyridinyl, isothiazolyl, tetrazolyl, benzimidazolyl, benzoxazolyl, benzothiazolyl, benzothiadiazolyl, benzoxadiazolyl, carbazolyl, indolyl, tetrahydroindolyl, azaindolyl, imidazopyridyl, quinzaolinyl, purinyl, pyrrolo[2,3]pyrimidyl, pyrazolo[3,4]pyrimidyl, benzo(b)thienyl, and the like. Examples of heteroaromatic compounds include, but are not limited to, furan, thiophene, pyrrole, pyridine, benzaldehyde, aniline, imidazole, thiazole, pyrimidine, oxazole, silole, azocine, and quinolone. These heteroaryl groups and heteroaromatic compounds may be optionally substituted with one or more substituents.

It will be appreciated that the above groups and/or compounds, as described herein, may be optionally substituted with any number of substituents or functional moieties. That is, any of the above groups may be optionally substituted. As used herein, the term “substituted” is contemplated to include all permissible substituents of organic compounds, “permissible” being in the context of the chemical rules of valence known to those of ordinary skill in the art. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and cannot be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like.

The following examples are intended to illustrate certain embodiments of the present invention, but do not exemplify the full scope of the invention.

### EXAMPLE 1

This example describes a hydrodeoxygenation reaction catalyzed by a perovskite compound, lanthanum strontium cobaltite (La_{0.8}Sr_{0.2}CoO₃).

The catalytic hydrodeoxygenation reaction was carried out in a vapor-phase packed-bed down-flow reactor. The reactor comprised a stainless steel tube having an outer diameter (OD) of 0.95 cm and a wall thickness of 0.089 cm mounted in a single-zone furnace (Applied Test Systems, Series 3210, 850 W/115 V). The temperature was controlled by a temperature controller (Digi-Sense, model 68900-10) connected to a K-type thermocouple (Omega, model TJ36-CAXL-116u) mounted downstream in direct contact with the catalyst bed.

The perovskite compound, lanthanum strontium cobaltite, La_{0.8}Sr_{0.2}CoO₃, was obtained from Sigma Aldrich. The phase of the perovskite compound was confirmed by powder X-ray diffraction pattern. 1.7 g of the lanthanum strontium cobaltite compound was pelletized between 60-100 mesh, mixed with inert α-Al₂O₃ diluent, and placed in the middle of the furnace. The total volume of the catalyst bed was about 2 cm³.

Prior to the reaction, the reactor temperature was ramped at a rate of about 6 K min⁻¹ under N₂ until reaching the reaction temperature (523 K). Next, an oxygenate feed stream comprising anisole, a compound found in bio-oil (obtained from the pyrolysis of biomass), was delivered into the reactor via a capillary tube connected to a syringe pump (Harvard Apparatus, model 703005) and mixed with H₂ gas at the inlet of the reactor. The total pressure of the reaction was 1.013 bar (Pₐₘₛₒₗₑ=0.0098 bar, atmospheric H₂). The perovskite compound contact times were changed by adjusting the flow rates of the oxygenate feed stream to vary the weight hourly space velocity (WHSV). The flow rate of the oxygenate feed stream was typically 200 μl h⁻¹.

The reactor effluent lines were heated to 523 K to prevent condensation. The effluents were analyzed and quantified via an online gas chromatograph (GC) fitted with a DB-5 column (Agilent, 30 m×0.25 mm inner diameter (ID)×0.25 μm) and equipped with a mass selective detector (MSD, Agilent Technologies, model 5975 C) for identification and a flame ionization detector (FID, Agilent Technologies, model 7890 A) for quantification. The gas chromatograph parameters used for analysis were as follows: detector temperature 573 K, injector temperature 548 K, and split ratio 1:20. The initial and final oven temperatures were 323 and 523 K, with a ramp of 10 K min⁻¹.

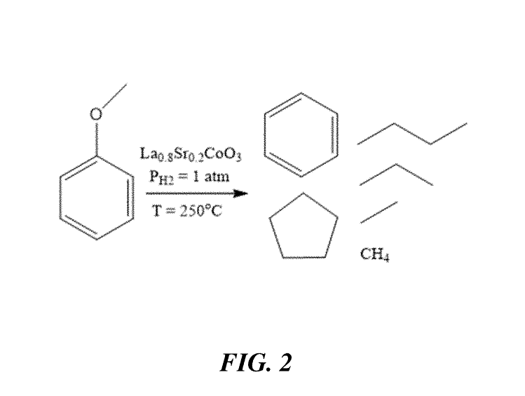

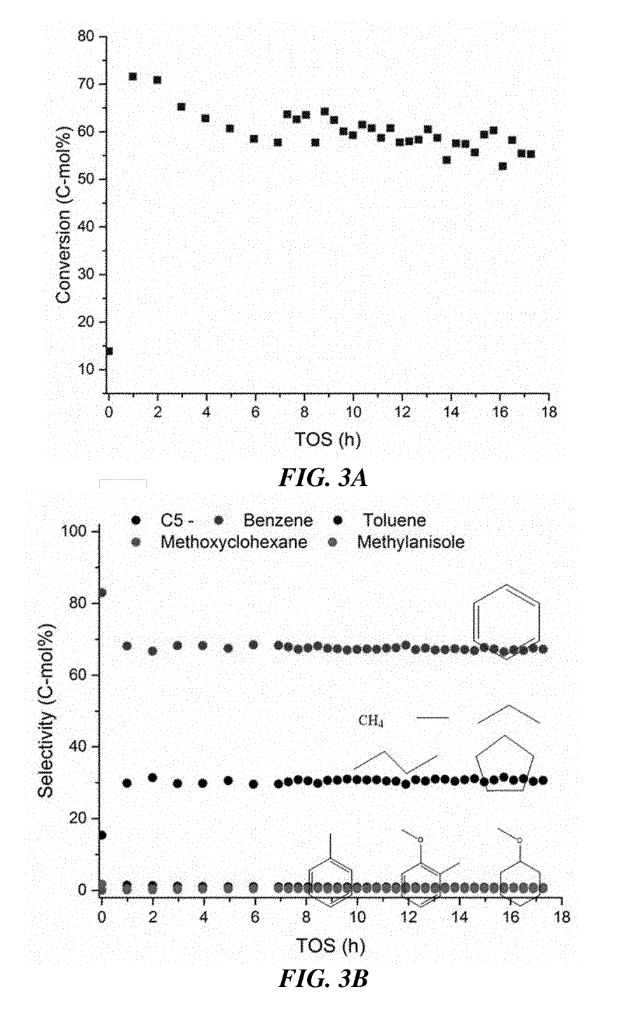

La_{0.8}Sr_{0.2}CoO₃ was demonstrated to be active for the hydrodeoxygenation of anisole. An exemplary reaction illustrating the hydrodeoxygenation of anisole to deoxygenated products, including benzene, cycloaliphatic hydrocarbons (e.g., cyclopentane), and aliphatic hydrocarbons (e.g., butane, propane, ethane, methane), is shown in FIG. 2. FIG. 3A shows a plot of conversion (carbon-mol %) as a function of time-on-stream (hours) that indicates that La_{0.8}Sr_{0.2}CoO₃ maintained greater than 50% conversion over a period of 18 hours. FIG. 3B shows plots of selectivity (carbon-mol %) for benzene (top), C₁-C₅ alkanes (middle), and substituted aromatic and cycloaliphatic hydrocarbons, including toluene, methoxycyclohexane, and methylanisole (bottom). As shown in FIG. 3B, anisole was converted to benzene with a selectivity of about 70 carbon-mol %. Other observed products were formed by hydrocracking of the intermediates to aliphatic hydrocarbons (e.g., C1-O5 alkanes) and cycloaliphatic hydrocarbons (e.g., cyclopentane).

### EXAMPLE 2

This example describes a hydrodenitrogenation (HDN) reaction catalyzed by a perovskite compound, lanthanum strontium cobaltite (La_{0.8}Sr_{0.2}CoO₃).

The experimental procedures of Example 1 were followed, except that 2 g of the perovskite compound, La_{0.8}Sr_{0.2}CoO₃, were pelletized, and a feed stream comprising pyridine, a model compound for the nitrogen-containing aromatics found in crude oil, was co-fed into the reactor in addition to a feed stream comprising anisole. In addition, the temperature was 200□, and the total pressure was 1.013 bar (0.0098 bar Pₐₙᵢₛₒₗₑ, 0.001 bar P_{pyridine,} balance H₂).

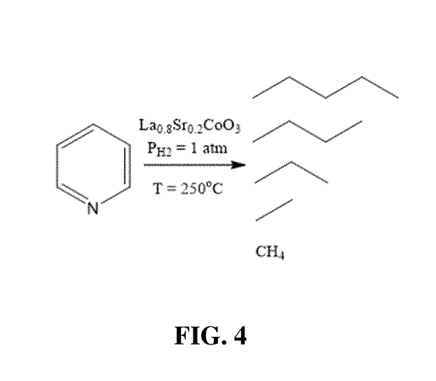

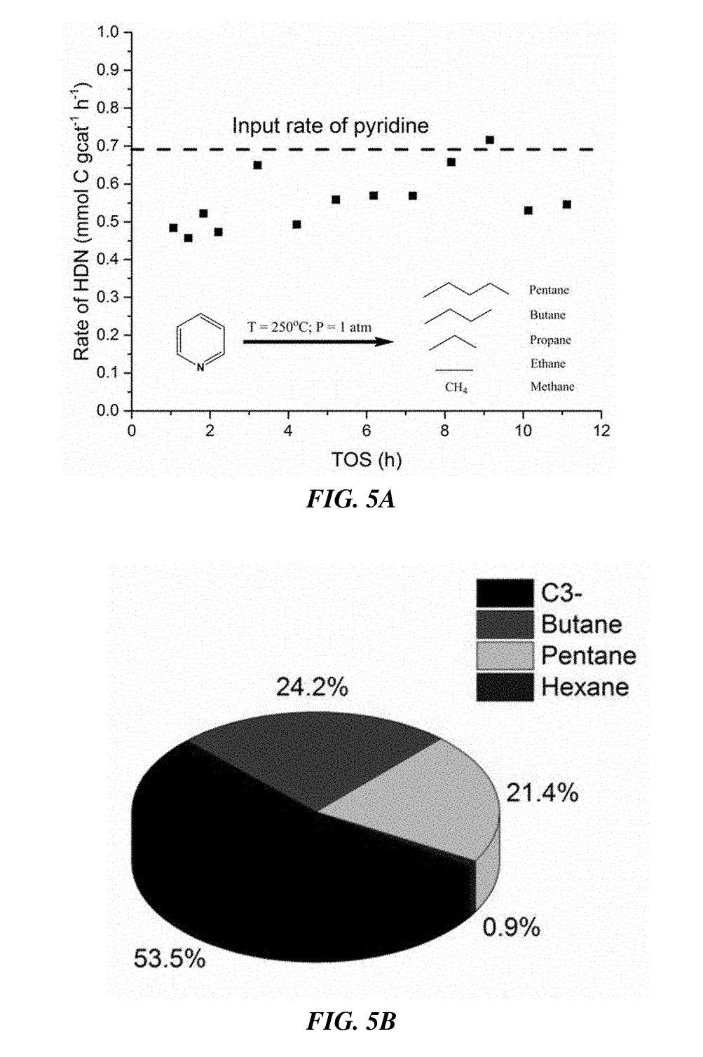

La_{0.8}Sr_{0.2}CoO₃ was demonstrated to be active for the hydrodenitrogenation of pyridine. An exemplary reaction illustrating the hydrodenitrogenation of pyridine to hydrocarbon products, including C₁-C₅ alkanes, is shown in FIG. 4. FIG. 5A shows a plot of rate of HDN products (mmol C gcat⁻¹h⁻¹) as a function of time-on-stream (hours). This plot shows that the rate of HDN products corresponded to 70-100% of the input rate of pyridine (about 9.7 carbon-mol %). FIG. 5B shows a pie chart indicating the selectivity of cracked products, including pentane, butane, propane, ethane, and methane. As shown in FIG. 5B, the selectivity of C₁-C₃ alkanes was 53.5 carbon-mol %, the selectivity of butane was 24.2 carbon-mol %, and the selectivity of pentane was 21.4 carbon-mol %. Less than 1 carbon-mol % (0.9 carbon-mol %) was contributed by cracking from methoxycyclohexane to hexane.

### EXAMPLE 3

This example describes a hydrodeoxygenation reaction catalyzed by a perovskite compound, lanthanum nickelate (LaNiO₃).

LaNiO₃ was synthesized by a sol-gel method. 1 mmol each of La³⁺and Ni²⁺ions (in their nitrate form) were solubilized in 1 g of water. Next, 2 mmol of citric acid corresponding to the total number of cations were added to the solution followed by ethylene glycol in the molar ratio of ethylene glycol:citric acid=4:1. The solution was heated to 115° C. for 12 hours until a gel was formed. The gel was dried at 100° C. for 12 hours and calcined at a temperatures greater than or equal to 700° C. for 6 hours. The formation of the perovskite compound was confirmed from powder X-ray diffraction patterns. The experimental hydrodeoxygenation procedures of Example 1 were followed, except that 500 mg of LaNiO₃ were pelletized.

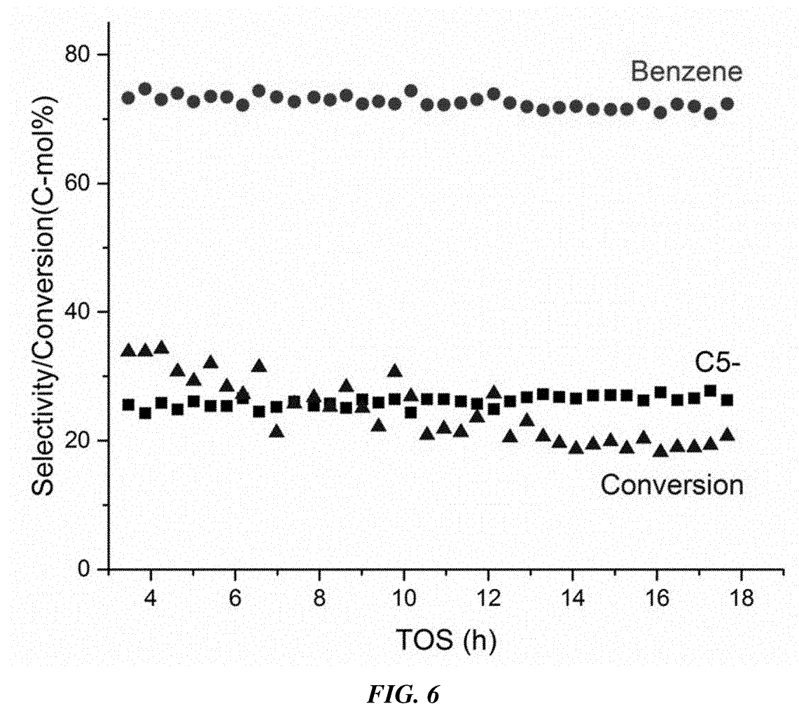

LaNiO₃ was demonstrated to be active for the hydrodeoxygenation of anisole. FIG. 6 shows a plot of selectivity/conversion (carbon-mol %) as a function of time-on-stream (hours). This plot shows that anisole was converted to benzene with a selectivity of about 70 carbon-mol %. Aliphatic hydrocarbons (e.g., C₁-O₅ alkanes) and cycloaliphatic hydrocarbons (e.g., cyclopentane) were formed by hydrocracking with a selectivity of greater than 20 carbon-mol %.

### EXAMPLE 4

This example describes a hydrodeoxygenation reaction catalyzed by a perovskite compound, lanthanum strontium cobaltite (La_{0.75}Sr_{0.25}CoO₃).

La_{0.75}Sr_{0.25} CoO₃ was synthesized by a sol-gel method. 0.75 mmol of La³⁺, 0.25 mmol of Sr²⁺, and 1 mmol of Co³⁺ions were solubilized in 1 g of water. Next, 2 mmol of citric acid corresponding to the total number of cations were added to the solution, followed by ethylene glycol in the molar ratio of ethylene glycol:citric acid=4:1. The experimental hydrodeoxygenation procedures of Example 1 were followed, except that 500 mg of

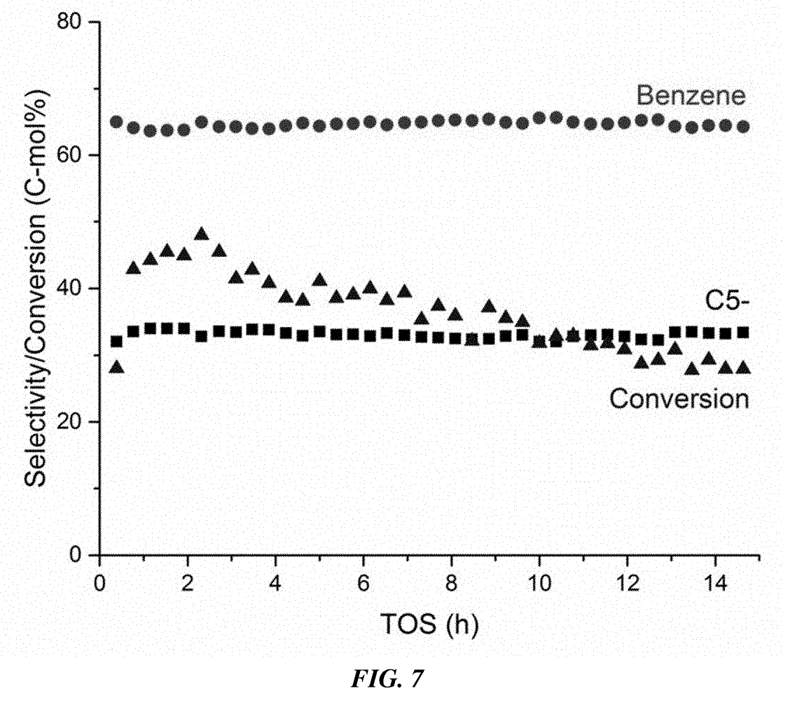

La_{0.75}Sr_{0.25}CoO₃ were pelletized. La_{0.75}Sr_{0.25}CoO₃ was demonstrated to be active for the hydrodeoxygenation of anisole. FIG. 7 shows a plot of selectivity/conversion (carbon-mol %) as a function of time-on-stream (hours). This plot shows that anisole was converted to benzene with a selectivity of about 65 carbon-mol %. Aliphatic hydrocarbons (e.g., C₁-C₅ alkanes) and cycloaliphatic hydrocarbons (e.g., cyclopentane) were formed by hydrocracking with a selectivity of greater than 30 carbon-mol %.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Having thus described several aspects of at least one embodiment, it is to be appreciated that various alterations, modifications, and improvements will readily occur to those skilled in the art. Such alterations, modifications, and improvements are intended to be within the spirit and scope of the present disclosure. Accordingly, the foregoing description and drawings are by way of example only.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc. in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
contacting a perovskite compound with a heteroatom-containing compound in the presence of H₂, wherein the perovskite compound catalyzes hydrogenolysis of the heteroatom-containing compound to produce one or more hydrocarbon products.

2. The method of claim 1, wherein the perovskite compound has the formula A₁₋ₓBₓDO₃, wherein:
A comprises a lanthanide;
B comprises an alkaline earth metal;
D comprises a transition metal; and
x is greater than or equal to 0 and less than or equal to 1.

3. The method of claim 1, wherein the heteroatom-containing compound comprises N, O, and/or S.

4. The method of claim 1, wherein hydrogenolysis comprises hydrodeoxygenation, hydrodenitrogenation, and/or hydrodesulfurization.

5. The method of claim 2, wherein A comprises La.

6. The method of claim 2, wherein B comprises Mg, Ca, Sr, and/or Ba.

7. The method of claim 6, wherein B comprises Sr.

8. The method of claim 2, wherein D comprises Cr, Mn, Fe, Co, and/or Ni.

9. The method of claim 8, wherein D comprises Co and/or Ni.

10. The method of claim 2, wherein x is 0, 0.2, 0.25, 0.5, 0.75, or 1.0.

11. The method of claim 1, wherein the perovskite compound is La_{0.8}Sr_{0.2}CoO₃, La_{0.75}Sr_{0.25}CoO₃, La_{0.25}Sr_{0.75}CoO₃, La_{0.25}Sr_{0.75}CoO₃, LaNiO₃, LaFeO₃, or SrCoO₃.

12-13. (canceled)

14. The method of claim 1, wherein the one or more hydrocarbon products comprise an aromatic hydrocarbon, and wherein the yield of the aromatic hydrocarbon is at least about 50 carbon-mol %.

15. (canceled)

16. The method of claim 1, wherein the one or more hydrocarbon products comprise one or more alkanes.

17. The method of claim 16, wherein the yield of the one or more alkanes is at least about 50 carbon-mol %.

18. (canceled)

19. The method of claim 1, wherein the contacting step is performed at a temperature of about 400° C. or less.

20. (canceled)

21. The method of claim 1, wherein the H₂ has a pressure of about 50 atm or less.

22. (canceled)

23. A method, comprising:
introducing a first feed stream comprising a heteroatom-containing compound into a reactor comprising a catalyst bed comprising a perovskite compound;
introducing a second feed stream comprising H₂ into the reactor;
flowing the first feed stream and the second feed stream, or a mixed feed stream comprising a mixture of the first feed stream and the second feed stream, through the reactor such that the first feed stream, the second feed stream, and/or the mixed feed stream directly contact the catalyst bed, wherein the perovskite compound catalyzes a hydrogenolysis reaction to produce one or more product streams comprising one or more hydrocarbon compounds.

24. The method of claim 23, wherein the hydrogenolysis reaction is a hydrodeoxygenation reaction, a hydrodenitrogenation reaction, and/or a hydrodesulfurization reaction.

25-28. (canceled)

29. The method of claim 23, wherein the perovskite compound has the formula A₁₋ₓBₓDO₃, wherein:
A comprises a lanthanide;
B comprises an alkaline earth metal;
D comprises a transition metal; and
x is greater than or equal to 0 and less than or equal to 1.

30-39. (canceled)

40. A system, comprising:
a first feed stream comprising a heteroatom-containing compound;
a second feed stream comprising H_{2;}
a perovskite compound; and
one or more product streams comprising one or more hydrocarbon compounds.

41-60. (canceled)
